(19) United States
(12) Patent Application Publication
(10) Pub. No.: US 2004/0077104 A1
(43) Pub. Date: Apr. 22, 2004

(54) Antigen detection device

(76) Inventors: Valentino Montegrande, Coto De Caza, CA (US); Kevin S. Montegrande, San Francisco, CA (US)

Correspondence Address: LAW OFFICES OF ERIC KARICH, 2807 ST. MARK DR., MANSFIELD, TX, 76063, US

(21) Appl. No.: 10/616,483

(22) Filed: Jul. 9, 2003

Related U.S. Application Data
(60) Provisional application No. 60/394,596, filed on Jul. 9, 2002.

(51) Int. Cl. G01N 33/551

(52) U.S. Cl. 436/524

(57) ABSTRACT

An antigen detection device for detecting the presence of an antigen in a fluid sample has a substrate micro-fabricated to define an antigen detection well. An antibody is disposed in the antigen detection well. The antibody is specific for the antigen being tested. An electrode positioned adjacent the antigen detection well is used to detect conductivity, which is turn can be used to determine whether the antibody is binding to an antigen.

### CROSS-REFERENCE TO RELATED APPLICATIONS

This application for a utility patent claims the benefit of U.S. Provisional Application No. 60/394,596, filed Jul. 9, 2002.

### STATEMENT REGARDING FEDERALLY SPONSORED RESEARCH

Not Applicable

### BACKGROUND OF THE INVENTION

1. Field of the Invention

This invention relates generally to diagnostic test kits, and more particularly to an antigen detection device that includes a substrate micro-fabricated to define a mesoscale flow system for detecting the presence of an antigen in a fluid sample.

2. Description of Related Art

The following art defines the present state of this field:

Wilding et al., U.S. Pat. No. 5,866,345, teaches an apparatus for the detection of an analyte using a MEMs-type chip. The device is microfabricated to include an inlet well, several sample flow channels, and an outlet well. The sample flow channels are coated with a binding moiety suitable for conducting fluorescent assays.

Herron, U.S. Pat. No. 6,242,267, teaches an apparatus for rapidly analyzing samples for analytes of interest by an immunofluorescence assay. The apparatus includes a sample test cartridge having a plurality of wells full of pre-loaded reagents. At least one of the wells receives a test sample, a second well receives a high control sample, and a third well receives a low control sample. The plurality of wells are coated with capture molecules that fluoresce when they bind with the analyte and are illuminated with light passing through a waveguide surface.

Parce et al., U.S. Pat. No. 6,156,181, teaches a microfabricated fluid transport device that is constructed from polymeric substrates.

The construction of microfluidic devices is discussed in Dubrow et al., U.S. Pat. No. 6,153,073. The reference teaches improved channel and reservoir geometries, as well as the use of the devices in the analysis of fluid born materials.

Various references teach tests for allergies, including the following:

Nutron, WO 93/25904, teaches a method for testing for an allergy to an antigen by exposing a blood sample to the antigen and measuring the degranulation of one or more of the granulocyte populations in the sample and comparing the measurement to a control.

Skov et al., U.S. Pat. No. 5,041,390, teaches an allergy test wherein the allergic response is measured by testing the amount of histamine in a sample. The histamine is determined through the use of a histamine binding agent such as glass fibers in a suitable binder.

Irsch et al., U.S. Pat. No. 5,916,818, teaches method for diagnosing allergen hypersensitivitiy by testing for allergen-binding cells. A population of blood cells from a blood sample is subjected to magnetic cell sorting to enrich the population of allergen-binding cells.

Andersson, U.S. Pat. No. 6,046,010, teaches a process for in vitro analysis of allergenic substances. Blood cells are cultivated in serial dilutions of the substances and cell proliferation is measured and the presence of cytokines is measured.

Lilius et al., U.S. Pat. No. 5,858,690, teaches a method for detecting allergies by measuring the receptor expression of phagocytic cells of peripheral blood.

Additional patents of interest include U.S. Pat. No. 6,207,367 B1, and U.S. Pat. No. 5,993,634, copies of which are enclosed.

The above-described references are hereby incorporated by reference in full.

The prior art teaches an apparatus for the detection of an analyte using a microfabricated device. However, the prior art does not teach a microfabricated device that tests for the presence of an analyte using electrode sensors positioned within each of a plurality of microfabricated flow channels. The present invention fulfills these needs and provides further related advantages as described in the following summary.

### SUMMARY OF THE INVENTION

The present invention teaches certain benefits in construction and use which give rise to the objectives described below.

The present invention provides an antigen detection device for detecting the presence of an antigen in a fluid sample. The antigen detection device has a substrate micro-fabricated to define an antigen detection well. An antibody is disposed in the antigen detection well. The antibody is specific for the antigen being tested. An electrode positioned adjacent the antigen detection well is used to detect conductivity, which is turn can be used to determine whether the antibody is binding to an antigen.

A primary objective of the present invention is to provide an apparatus for the detection of an analyte using a microfabricated device having advantages not taught by the prior art.

Another objective is to provide an apparatus that includes electrode sensors positioned within each of a plurality of microfabricated flow channels for the purposes of detecting the presence of the analyte electronically.

A further objective is to provide an apparatus that is inexpensive to manufacture and easy to use.

Other features and advantages of the present invention will become apparent from the following more detailed description, taken in conjunction with the accompanying drawings, which illustrate, by way of example, the principles of the invention.

### BRIEF DESCRIPTION OF THE DRAWING

The accompanying drawings illustrate the present invention. In such drawings:

FIG. 1 is a top plan view of the preferred embodiment of the present invention, an antigen detection device for detecting the presence of an antigen or reagent in a fluid sample;

FIG. 2 is a perspective view of one of a plurality of antigen detection wells that are disposed on a top surface of the antigen detection device;

FIG. 3 is a sectional view taken along line 3-3 in FIG. 2;

FIG. 4 is an exploded perspective view of another embodiment of the antigen detection device, illustrating how a substrate is bonded to an electronic interface chip to form the antigen detection device;

FIG. 5 is a bottom perspective view thereof; and

FIG. 6 is a perspective view of the antigen detection device mounted upon a handle for insertion into an electronic reader apparatus.

### DETAILED DESCRIPTION OF THE INVENTION

The above described drawing figures illustrate the invention, an antigen detection device for detecting an antigen in a fluid sample.

Substrate

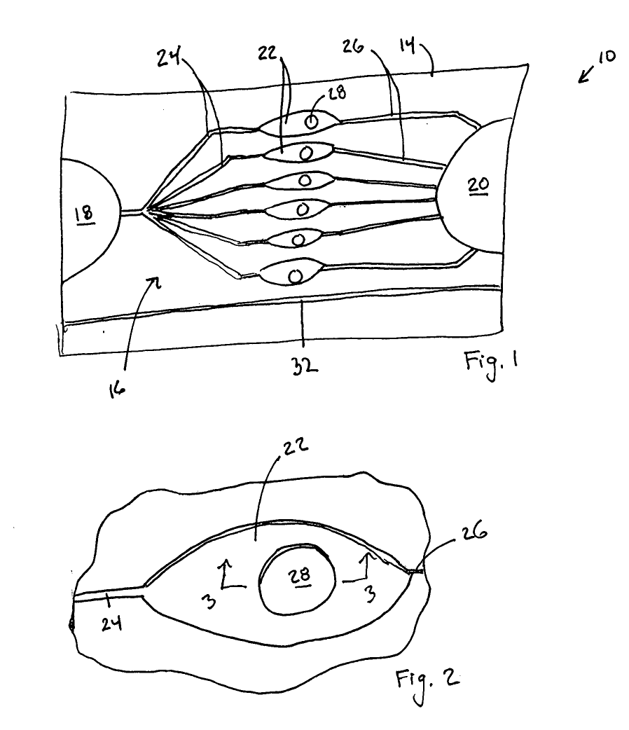

As shown in FIG. 1, the antigen detection device includes a substrate, preferably constructed of silicon, that is about 3 mm thick (generally about 1-5 mm) and approximately 1.15 centimeters wide and 1.64 centimeters long (generally about 1-5 cm); however, these parameters can vary depending upon the needs of the user and the manufacturer. The substrate is micro-fabricated, using techniques known in the art, to define a mesoscale flow system.

Mesoscale Flow System

As shown in FIG. 1, the mesoscale flow system includes an input well; an outlet well; a plurality of antigen detection wells; a plurality of input flow channels in fluid communication with input well and each of the plurality of antigen detection wells; and a plurality of outlet flow channels in fluid communication with outlet well and each of the plurality of antigen detection wells.

As shown in FIG. 2, each of the plurality of antigen detection wells is adapted to contain an antibody for specifically binding the antigen. For purposes of this application, the term antibody should be construed to include any form of binding moiety, whether alone, in combination, or as part of a reagent. The plurality of antibodies, each specific to a regionally important antigen, may be introduced into each of the plurality of antigen detection wells. The antibodies can be introduced in the factory, or the substrate can be delivered to the customer as blanks and the customer can then add any of the types of antibodies that are desired. This is helpful because different geographical regions contain different antigens, and it is easier to add the necessary antibodies at the time of use rather than manufacture many different versions of the device.

In an alternative embodiment, the antibodies may be immobilized in the antigen detection wells during the manufacture of the device. Immobilization options include but are not limited to physical absorption or chemical attachment to the surface of the flow system or to a solid phase reactant such as a polymeric bead disposed in the detection region. Those skilled in the art can devise many different options for associating the antibodies with the antigen detection wells, and such alternative embodiments should be considered as included in the present disclosure.

The surfaces of the mesoscale detection channels in the silicon substrates can be chemically activated and reacted with a protein, lipid, polysaccharide or other macromolecule to form a coated surface in the mesoscale flow channels. Techniques for the chemical activation of silaceous surfaces are available in the art. There are a number of techniques in the art for attaching biomolecules to silicon. For example, enzymes may be immobilized on silicon devices via entrapment in a photo-crosslinkable polyvinyl alcohol. A hydrophobic bilayer glycerol monooleate coating may be fabricated on a silicon substrate. Protein conjugation and immobilization techniques known in the art may be adapted for use with activated silaceous surfaces. Known chemistries in the art may be adapted for use in attaching biomolecules to coated or uncoated silicon channel surfaces. A binding moiety such as an antigen binding protein, a polynucleotide probe, or one of a ligand/receptor pair may be attached to the silicon channel surfaces. The surface coated mesoscale flow systems can be utilized in any of a wide range of available binding assays known in the art such as immunoassays, enzymatic assays, ligand/binder assays, polynucleotide hybridization assays, and cell surface binding assays. The detection of cellular or macromolecular antigens can be implemented by selecting the appropriate binding moiety coated on the surface of the detection region. Such techniques are described in greater detail in Wilding et al., U.S. Pat. No. 5,866,345, hereby incorporated by reference in full.

In one embodiment, each of the plurality of antigen detection wells has an antibody containment basin in which the antibodies are contained or bound, although this feature is not used in some embodiments. The antibodies may be immobilized within the detection region. As disclosed herein, mesoscale detection systems may be used in a wide range of rapid tests for various antigens. The devices may be fabricated with two or more mesoscale flow systems which comprise two or more different detection regions containing binding moieties for different antigens, allowing two or more assays to be conducted simultaneously. At the conclusion of the assay the devices can be discarded due to the inexpensive nature of the device.

Mesoscale devices having a mesoscale flow system can be designed and fabricated in large quantities from a number of solid substrate materials; however, silicon is preferred because of the enormous body of technology permitting its precise and efficient fabrication. Alternative embodiments include polymers such as polytetrafluoroethylenes.

The mesoscale flow system can be fabricated inexpensively in large quantities from a silicon substrate by any of a variety of micro-machining methods known to those skilled in the art. The micromachining methods available include film deposition processes such as spin coating and chemical vapor deposition, laser fabrication or photolithographic techniques such as UV or X-ray processes, or etching methods including wet chemical processes or plasma processes. Flow channels of varying widths and depths can be fabricated with mesoscale dimensions, with cross-sectional dimensions preferably between 0.1 to 500 μm.

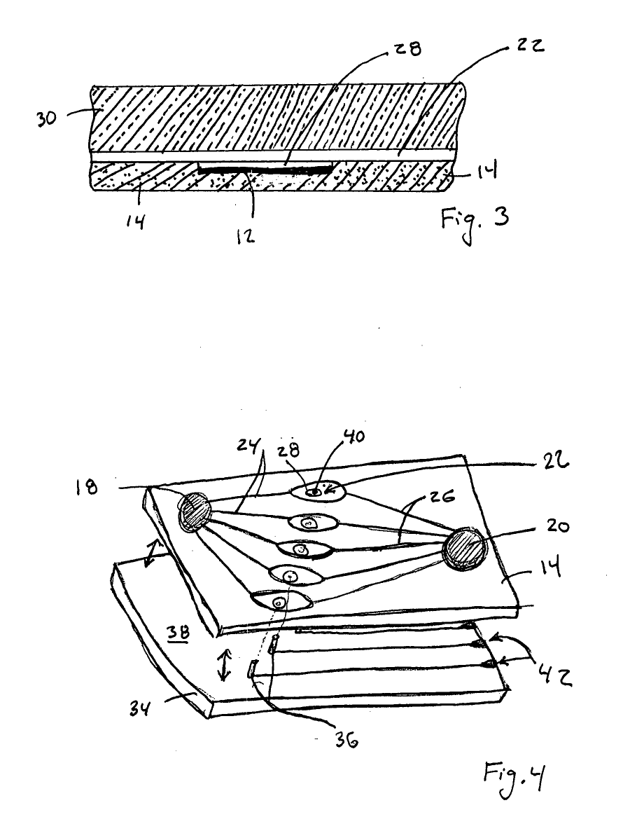

As shown in FIG. 3, the silicon substrate can be covered and sealed with a cover that is bonded, preferably with anodic bonding, to the substrate. While the cover can be made of glass, an equivalent material such as silicon or quartz may be used.

Detection

The device further includes a means for detecting whether the antibody is binding to an antigen in at least one of the plurality of antigen detection wells. The means for detecting the binding of the antigen to the antibodies in the detection region can be detected by any of a number of methods including monitoring the electrical conductivity of fluid samples in the device as disclosed herein or by optical detection.

In one embodiment, as shown in FIG. 4, the binding of the antigen can also be detected by sensing electrical conductivity at some region within the device. The conductivity of the fluid sample in each of the plurality of antigen detection wells can be measured in order to detect changes in electrical properties upon antigen binding to binding moieties in the detection region. In this embodiment, the device includes an electronic interface chip that measures the changes in electrical properties.

The electronic interface chip is preferably constructed of silicon and preferably includes a plurality of electrical probes disposed on a top surface of the electronic interface chip. Each of the plurality of electrical probes are shaped and positioned to fit through a matching one of a plurality of probe apertures of the substrate into one of the plurality of antigen detection wells. Each of the plurality of electrical probes is electrically connected to one of a plurality of electrical contacts, which are adapted to interface with an electronic reader apparatus for reporting the results of the test.

The electronic reader apparatus, shown in FIG. 6, operates to establish an electrical connection with the electrode or electrodes of the substrate, take readings, and compute with a computer whether the antibodies are binding with the antigens of the fluid sample.

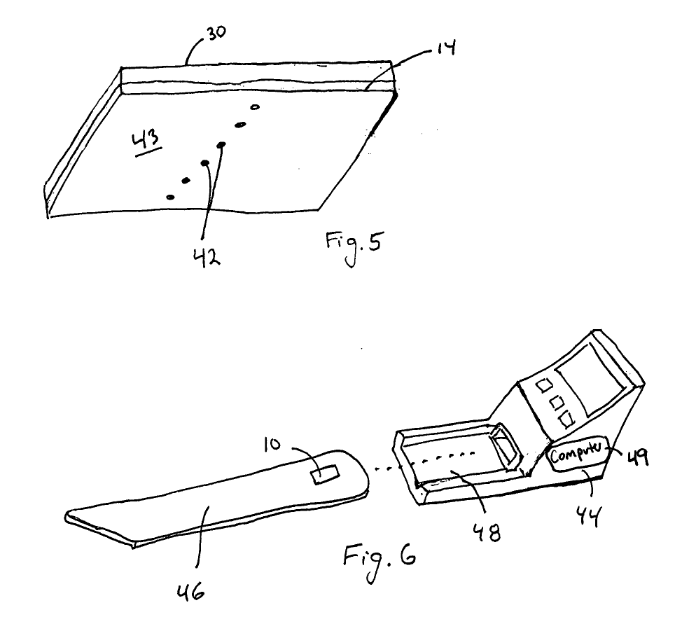

In an alternative embodiment, as shown in FIG. 5, the plurality of electrical probes extend through the substrate to form the plurality of electrical contacts on a bottom surface of the substrate.

The electronic interface chip is preferably bonded to the substrate for positioning the electrical probes through the probe apertures. In the preferred embodiment, as shown in FIG. 6, the substrate and the electronic interface chip are then mounted upon a handle to facilitate handling of the device. The handle is then inserted into a nesting site of the electronic reader apparatus, thereby bringing the plurality of contacts into operable contact with the electronic reader apparatus for reporting the results of the test.

In one embodiment, the binding of an antigen to the antibodies in the detection region may also be detected optically using a detectable label, such as a fluorescent or luminescent molecule that is bonded to either the antigen or the binding moiety. The fluorescent or luminescent label in the detection region can be detected when the antibody binds with the antigen. Alternatively, a second labeled substance, such as a fluorescent labeled antibody can be delivered through the flow system to bind to the bound antigen/binding moiety complex in the detection region to produce a “sandwich” including an optically detectable moiety whose presence is indicative of the presence of the antigen. For example, immunofluorescent labels reported in the prior art may be utilized. The device preferably includes a reference fiber optic associated with the substrate. The reference fiber optic is used as a control for calibrating the optical reader device (not shown) that is used to interpret the results. Since such technology is readily understood by those skilled in the art once given the teachings of this invention, it is not described in greater detail herein. In the situation involving LEDs (light-emitting diodes) or fluorescent labeled reagents, the fluorescent light source or LEDs are directed in a pin point focus to penetrate the wells containing the antibody/antibody reactions. Receiver platens or photocells receive the light dispersed or absorbed by the binding reactants an can be directly correlated to the degree of response of the antigen.

While the invention has been described with reference to at least one preferred embodiment, it is to be clearly understood by those skilled in the art that the invention is not limited thereto. Rather, the scope of the invention is to be interpreted only in conjunction with the appended claims.

The elements listed above may be linked with the drawings using the below-provided element list:

antigen detection device 10

antibody 12

substrate 14

mesoscale flow system 16

input well 18

outlet well 20

plurality of antigen detection wells 22

plurality of input flow channels 24

plurality of outlet flow channels 26

antibody containment basin 28

cover 30

reference fiber optic 32

electronic interface chip 34

plurality of electrical probes 36

top surface 38

plurality of probe apertures 40

plurality of electrical contacts 42

bottom surface 43

electronic reader apparatus 44

handle 46

nesting site 48

computer 49

What is claimed is:

1. An antigen detection device for detecting the presence of an antigen in a fluid sample, the antigen detection device comprising:
a substrate micro-fabricated to define am antigen detection well;
an antibody disposed in the antigen detection well, the antibody being specific for the antigen being tested; and
an electrode positioned adjacent the antigen detection well for detecting whether the antibody is binding to an antigen.

2. The antigen detection device of claim 1 wherein the electrode is micro-fabricated as part of the substrate.

3. The antigen detection device of claim 1 wherein a plurality of the antigen detection wells are provided on the substrate.

4. The antigen detection device of claim 1 wherein the antigen detection well includes an antibody containment basin.

5. The antigen detection device of claim 1 further comprising an electronic reader apparatus adapted for making an electronic connection with the electrode, the electronic reader apparatus having a computer programmed to determine whether the antigen is binding with the antibody.

6. An antigen detection device for detecting the presence of an antigen in a fluid sample, the antigen detection device comprising:
a substrate micro-fabricated to define a mesoscale flow system that includes:
an input well;
an outlet well;
a plurality of antigen detection wells;
a plurality of input flow channels in fluid communication with the input well and each of the plurality of antigen detection wells; and
a plurality of outlet flow channels in fluid communication with outlet well and each of the plurality of antigen detection wells;
at least one test reagent containing an antibody that is specific for the antigen being tested; and
a means for detecting whether the antibody is binding to an antigen in at least one of the plurality of antigen detection wells.

7. The antigen detection device of claim 6 wherein each of the at least one test reagents is disposed within one of the plurality of antigen detection wells.

8. The antigen detection device of claim 6 wherein the means for detecting is an electrode positioned adjacent the antigen detection well for detecting whether the antibody is binding to an antigen.

9. The antigen detection device of claim 6 wherein each of the plurality of antigen detection wells includes an antibody containment basin.

10. The antigen detection device of claim 6 further comprising an electronic reader apparatus adapted for making an electronic connection with the electrode, the electronic reader apparatus having a computer programmed to determine whether the antigen is binding with the antibody.

11. A method for detecting the presence of an antigen in a fluid sample, the method comprising the steps of:
providing a substrate micro-fabricated to define an antigen detection well with an electrode positioned adjacent to the antigen detection well;
binding an antibody specific to the antigen to the antigen detection well;
adding the fluid sample to the antigen detection well;
measuring the electrical conductivity at the electrode; and
determining whether the antibody is binding to an antigen in the antigen detection well based upon the measured conductivity.
